(19) United States
(12) Patent Application Publication
(10) Pub. No.: US 2024/0268679 A1
(43) Pub. Date: Aug. 15, 2024

(54) NON-INVASIVE DEVICE FOR QUICKLY ESTIMATING ANAEMIA

(71) Applicant: Universita' degli Studi di Bari Aldo Moro, Bari (IT)

(72) Inventors: Giovanni DIMAURO, Bari (BA) (IT); Rosalia MAGLIETTA, Bari (BA) (IT)

(73) Assignee: Universita' degli Studi di Bari Aldo Moro, Bari (IT)

(21) Appl. No.: 18/568,963

(22) PCT Filed: Jun. 29, 2022
(86) PCT No.: PCT/IB2022/056041

§ 371 Date: Dec. 11, 2023

(30) Foreign Application Priority Data
Jun. 29, 2021 (IT) 102021000017078

(51) Int. Cl. A61B 5/00

(52) U.S. Cl. CPC A61B 5/0082; A61B 5/0077; A61B 5/6821; A61B 5/7267; A61B 5/742

(57) ABSTRACT

The present invention relates to a method and a corresponding device for quickly and non-invasively estimating the state of anaemia in a subject, thanks to capturing and subsequently processing a digital image of the palpebral conjunctiva.

### SUMMARY OF THE INVENTION

The present invention relates to a method and a corresponding device for quickly and non-invasively estimating the state of anaemia in a subject, thanks to capturing and subsequently processing a digital image of the palpebral conjunctiva.

### BACKGROUND ART

Anaemia is the most common blood disorder that can be found in the population, as the WHO estimates that around 2 billion people worldwide are affected by it. Anaemia is defined as the pathological reduction in the number of circulating red blood cells in the blood or as the decrease in the concentration of haemoglobin below normal levels and has, as a direct consequence, a reduced capacity of the blood to carry oxygen. In most cases, this condition sets in slowly and progressively and is accompanied by symptoms such as a feeling of tiredness, weakness, shortness of breath and/or poor physical exercise ability.

The anaemia condition can be determined by several factors, which may have a nutritional-type origin (e.g. iron, vitamin and/or mineral deficiency), an infectious-type origin (e.g. malaria, intestinal parasites) or a genetic-type origin (e.g. haemoglobinopathies). These factors may occur alone or, more frequently, in combination with each other.

In particular, the anaemia caused by iron deficiency is the most common nutritional deficiency and is also considered to be responsible also for the increase in morbidity and mortality in pre-school children and pregnant women.

In current medical practice, one of the most commonly employed parameters for the diagnosis of anaemia as well as for the subsequent determination of patients' response to treatment is the measurement of haemoglobin levels in the blood. In particular, subjects with a haemoglobin value below 13 g/dL or 12 g/dL, respectively, for male or female adult subjects are considered to be at risk of anaemia, although these thresholds are much discussed in the literature. Moreover, the thresholds vary with age, pregnancy status, cigarette smoking and other factors. Patients in the most critical conditions should repeat this test very frequently, by undergoing blood samples to evaluate the possible need for a transfusion if values fall too far below the alert level. The most accurate determination of the anaemia status, therefore, requires a venous blood sample and the use of dedicated laboratory instrumentation. It is therefore an invasive examination with a non-negligible cost, especially if one takes into account the healthcare personnel that must necessarily be involved.

In recent years, alternative approaches have also been proposed, which are mainly based on the observation and evaluation of the colour of the palpebral conjunctiva. These methods include, in the simplest case, visual evaluation by the treating physician who, based on his or her experience, can decide whether or not the patient is in a condition of severe anaemia. This method is widespread and used in medical practice especially in areas of the earth where carrying out laboratory investigations is difficult or too onerous. It is a quick and inexpensive method but it clearly brings with it numerous disadvantages, which are mainly related to the fact that it is a method that cannot in any case give reliable qualitative results and that is strongly influenced both by the experience of the physician performing the evaluation and by the quality and quantity of ambient light present at the time of the observation.

Other approaches, such as e.g. the one described in US2003002722, are based on the possibility of simultaneously capturing a digital image of the palpebral conjunctiva and a standard colour sample card with a camera, and then evaluating the shade of the conjunctiva in comparison to the standard thanks to the use of a personal computer equipped with software for image analysis. This method is certainly less influenced by the skill and experience of the operator than the previous method described above but, even in this case, some disadvantages remain which are mainly related to the fact that the method of US2003002722 can be influenced by ambient light and requires additional instrumentation (personal computer) and qualified personnel capable of manipulating the image in order to capture the measurement correctly.

The possibility of using a smartphone, and in particular its camera, to capture the image of the palpebral conjunctiva, in order to perform a determination of the level of possible anaemic pathology in a subject, was also proposed. IT102017000043624 describes the use of a particular spacer, which can be coupled to LEDs and electrical connectors placed on the outside of said spacer, to be connected to the same smartphone, which allows to at least partially decrease the influence of ambient light during the capture of the image of the palpebral conjunctiva. Said spacer must, however, be continuously assembled and disassembled from the main body of the smartphone and connected thereto through cables. The system and device described in IT102017000043624 therefore do not completely solve the problem of the influence of ambient light in the evaluation of the colour of the palpebral conjunctiva. In fact, the system described cannot be perfectly adapted to the different types of smartphones, nor to the specific facial characteristics of the subject whose haemoglobinaemia is to be evaluated. Moreover, said measuring system is complex and not self-sufficient, as it necessarily implies the use of a second external apparatus, i.e. the smartphone, without which it would not be able to function.

The need is therefore still felt to have a system that allows the evaluation of the haemoglobin value of the blood that is low-cost, can be easily carried and used even in areas of the world where clinical facilities are scarcely available in which to carry out laboratory investigations.

There is an equally strong need for a system that is easy to employ and carry, that allows the evaluation of the haemoglobin value in blood, that is not influenced by ambient light, that does not require specialised personnel for its use and, above all, that does not need to be coupled to a second or additional piece of equipment in order to function, but is self-sufficient.

There is also a need for a method that allows the evaluation of haemoglobin concentration in blood, that is not influenced by ambient light, that does not require specialised personnel for its implementation and that is based on the use of a simple and reliable device.

### OBJECTS OF THE INVENTION

It is an object of the present invention to provide a new device for quickly estimating the possible anaemia condition in a patient, which is non-invasive and inexpensive.

It is also an object of the present invention to provide a new device for mass screening to diagnose anaemia, especially in areas of the earth with limited economic resources and availability of analysis laboratories.

It is still the object of the present invention to provide a device that is capable of functioning autonomously and without the need for the aid of other external equipment or technologies and that can also be used without the need for specific training courses. Still another object of the present invention is the use of the new device to carry out a quick estimation of the haemoglobin concentration in a subject.

This and other purposes are achieved by the object of the present invention, which has as its subject matter a non-invasive device for estimating the haemoglobin concentration in a subject and the consequent evaluation of possible forms of anaemia.

### BRIEF DESCRIPTION OF THE FIGURES

FIG. 1: CAD drawing of the spacer/adapter of the device of the invention; profile, top view in transparency (a), with highlighted perimeter lines (b), solid (c).

FIG. 2: CAD drawing of the spacer/adapter of the device of the invention; profile, side view in transparency (a), with highlighted perimeter lines (b), solid (c). (1) is the side on which the subject's periocular area rests, (2) is the outer side.

FIG. 3: CAD drawing of the spacer/adapter of the device of the invention; profile, front view in transparency (a), with highlighted perimeter lines (b), solid (c). (1) is the side on which the subject's periocular area rests, (2) is the outer side.

FIG. 4: CAD drawing of the spacer/adapter of the device of the invention; three-dimensional view in transparency (a), with highlighted perimeter lines (b), solid (c). (1) is the side on which the periocular area of the subject rests, (2) is the outer side comprising a circular hole (3) for housing the macro lens.

FIG. 5: simplified circuit diagram of the connections of the electronic components of an embodiment of the device in accordance with the invention.

### DESCRIPTION OF THE INVENTION

Object of the present invention is a device which non-invasively and quickly allows to estimate the condition of possible anaemia in a subject, by means of the capture of an image of the palpebral conjunctiva and subsequent analysis of its colour hue and/or intensity by software integrated within the same device.

Object of the present invention is also to provide a method for evaluating the haemoglobin concentration in a subject and the corresponding evaluation of a possible form of anaemia in the same subject, which provides for the use of the device above. According to the present invention, the “estimation/measurement of haemoglobin concentration”, which can be directly obtained by means of the device and method according to the invention, can be correlated to the possible anaemia condition of the subject on whom the measurement is performed, thus allowing to evaluate whether or not an anaemia condition of the same subject is present.

The device that is the object of the present invention, mainly consists of:
a) a computerised processing unit, which implements the algorithms for the estimation of the haemoglobin concentration level based on the colour of an image, on which software is installed that uses image analysis algorithms to extract the information from the images and artificial intelligence algorithms to evaluate its colour characteristics and compare them with standard values encoded therein;
b) an imaging unit comprising a miniature video camera;
c) a control display for controlling the area, which is framed by the device, to be photographed;
d) an optical unit comprising at least one lens that allows images of the ocular area to be shot at close range;
e) a spacer/adapter comprising a hollow body having a supporting surface configured to fit the periocular area, by adapting perfectly to the shape of the frontal bone, the zygomatic bone and the maxillary bone of the subject to be evaluated and further allowing a cavity enclosed therein, shot by the video camera, to be isolated from the ambient light;
f) a lighting unit arranged within the spacer and combined with the cavity of the spacer, configured to illuminate an inner space of the cavity and which allows images to be captured with a light of constant intensity and known colour.

It should be noted that in the present document the terms “adapter” and “spacer” are used interchangeably as synonyms.

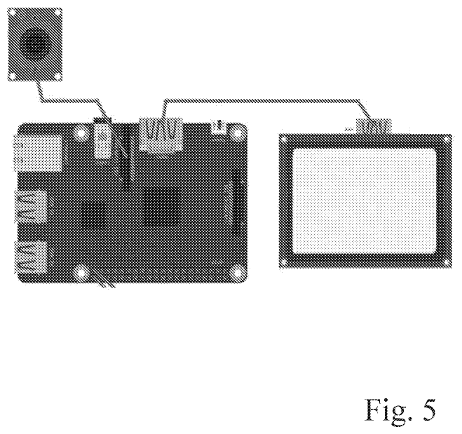

In the present document, the term “combined”, when related to a device element, refers to its possible mechanical coupling with the part to which it is assembled/connected. The term miniature video camera b) means an image sensor which can be connected to the computerised processing unit referred to in point a), e.g. by means of a cable, preferably connected to the CSI port as illustrated in FIG. 5. According to a preferred aspect of the invention, said miniature video camera has a resolution of 8 megapixels, 25 mm×23 mm×9 mm dimensions, fixed-focus lens, reproduction of 3280×2464 pixels static images and 1080p30, 720p60 and 640×480p90 video reproduction. Said miniature video camera of the imaging unit, referred to in point b), is intended for the capture of an image which comprises the area of the palpebral conjunctiva without the need for the capture of other reference images or a colour reference. The image is captured by the miniature video camera in the form of digital pixels stored by the system in RGB (standard Red, Green, Blue) colour space format. The same digital pixels are then transformed, by means of the software referred to in point a), to also be represented by means of the CIELAB and HSI colour spaces. The CIELAB colour space uses two dimensions (a* and b*) for colour representation (red-green and yellow-blue, respectively) and the dimension L* for brightness. The HSI colour space uses the H components (Hue, dominant colour perceived by the observer and is therefore related to the wavelength present in the light received by the video camera), S (Saturation, purity of the colour which is lesser the more the colour is diluted by the white component) and I (Intensity, amount of light received by the video camera).

It should be noted that the terms “video camera” and “camera” are used interchangeably as synonyms in the present document.

The algorithms, referred to in point a), allow the following operations to be performed:
the automatic or assisted selection on the image, captured by the miniature video camera referred to in point b) and shown on the display referred to in point c), of the entire area occupied by the palpebral conjunctiva or a part thereof, thus determining the identification of a specific portion of the same image;
the extraction, in the delimited area consisting of the specific portion of the previously selected image, of the values meanᵣ, mean_{g}, mean_{b}, mean_{L}, meanₐ*, mean_{b}*, HHR, E, B, G₁, G₂, G₃, G₄, G₅ (as described in the following experimental section) of the pixels contained therein;
the estimation of haemoglobin concentration depending on the values meanᵣ, mean_{g}, mean_{b}, mean_{L}, meanₐ*, mean_{b}*, HHR, E, B, G₁, G₂, G₃, G₄, G₅ corresponding to the pixels of the specific portion of the previously captured and selected image, as well as the patient's age and sex;
the reporting of the level of anaemia and the consequent indication to the patient that he/she is in a safe condition or that he/she has reached values considered to be alarming.

Said operations can be performed by the algorithms specially installed in, and executable by, the computer processing unit referred to in point a), on which the images captured by the miniature video camera are also stored as well as the values meanᵣ, mean_{g}, mean_{b}, mean_{L}*, meanₐ*, mean_{b}*, HHR, E, B, G₁, G₂, G₃, G₄, G₅ obtained by the extraction of the pixels, in addition to the age and sex of the patient and the estimation of the haemoglobin level. The device of the invention, thanks to the imaging unit and the computerised processing unit with the respective algorithms installed, is therefore capable of recording and measuring 14 parameters which, together with the characteristics of the patient (age and sex), constitute 16 distinct parameters that allow to predict, with a high degree of precision, the anaemia condition of the subject undergoing analysis.

The images captured, the patient's data and the values estimated by the device can be sent to the physician for a further check, either by using the same device or by means of a personal computer or other digital processing/communication device.

The display, referred to in point c), is preferably a touchscreen-type display, preferably 3.5 inch LED with a resolution of 320×480 pixels. As illustrated in FIG. 5, the display is operatively connected to the computerised processing unit referred to in point a), e.g. by means of an HDMI cable. According to an embodiment of the invention, said display is a 3.5 inch touch screen LED display for Raspberry Pi4/4B, model T-4BABS02, LCD type: TFT, LCD interface: SPI (supports SPI input up to 125 MHz) type of touch-screen: resistance, resolution: 320×480 pixels, absorption: 120 mA. The lens of the optical unit, referred to in point d), placed in the circular hole (3) highlighted in FIG. 4, is a macro lens, e.g. a 25 mm diameter lens, 10×. Said lens is mechanically coupled to the circular hole (3); advantageously, said mechanical coupling allows to keep the lens always in the same position and, consequently, to optimise the repeatability of the capture of the images.

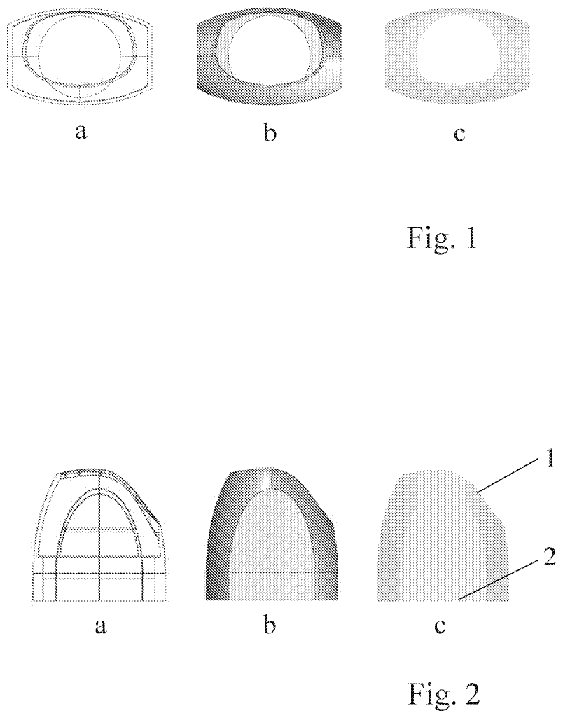

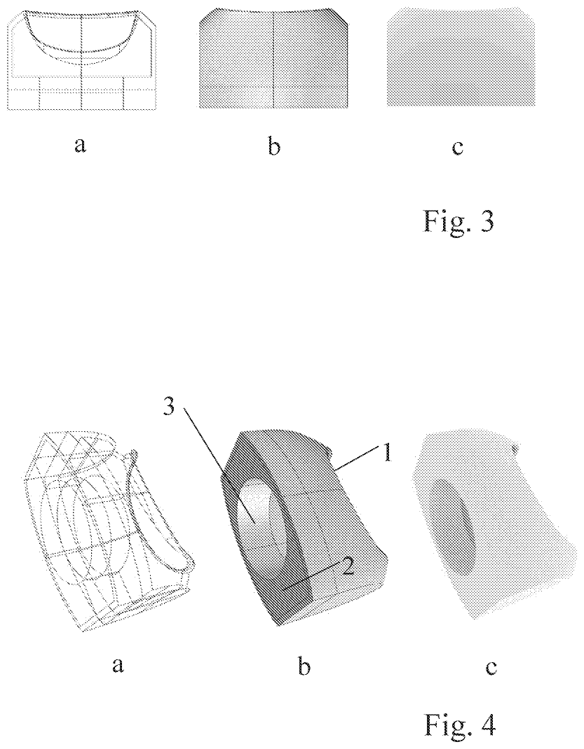

The spacer/adapter, referred to in point e) above, has specific characteristics which make it unique and particularly advantageous in its use, due to the fact that it allows the instrument to rest on the periocular area, by adapting perfectly to its shape and further allowing the area enclosed therein shot by the video camera to be isolated from the ambient light. Said spacer/adapter for capturing digital images of the palpebral conjunctiva is an object having shape and characteristics adapted to capture digital images of the human palpebral conjunctiva by means of a miniature video camera and a macro lens. Said spacer/adapter comprises a hollow body having a supporting surface and a cavity. Said supporting surface is configured, in use, to be placed in contact with the periocular area of the subject to be evaluated. The model has an original and ergonomic shape (FIGS. 1-4) in particular at the supporting surface which is shaped, i.e. configured, in such a way as to be adapted to the human orbital cavity and in particular to the shape of the frontal bone, the zygomatic bone and the maxillary bone. Preferably, said body of the spacer/adapter comprises a user surface opposite said supporting surface. Said user surface is configured to accommodate said optical unit placed in the circular hole (3) placed on the outer side (2) of the spacer/adapter, said display, as well as other electronic components of the device, such as the computerised processing unit and the imaging unit.

According to an aspect of the invention, in use the spacer/adapter is interposed between the back casing containing the electronic circuitry and the orbital cavity, by laying it on the latter. Its shape allows the geometric plane containing the palpebral conjunctiva to be aligned with the geometric plane containing the camera and therefore allows the optimal capture of digital images of the same conjunctiva in a non-invasive manner. In other words, the axis of the spacer/adapter, due to its special shape, is perpendicular to the plane containing the palpebral conjunctiva.

Moreover, the spacer/adapter, made of opaque material, allows the image of the palpebral conjunctiva to be captured from a fixed distance and excluding the influence of ambient illumination. These characteristics make it adapted to capture images of the palpebral conjunctiva for diagnostic purposes.

The lighting unit, referred to in point f), consists of one or more, preferably more than one, LEDs mounted inside the spacer/adapter, referred to in point e). Said LEDs may, e.g., be arranged in a circle around the macro lens. In particular, said lighting unit is combined with said cavity of the body of the spacer/adapter and is configured to illuminate a space inside the cavity, which, when the device is positioned in contact with the periocular area of the subject to be evaluated, is in the dark. Advantageously, the positioning of the LEDs mounted inside the spacer/adapter allows to minimise the areas of reflection, typically white in colour, due to moisture in the conjunctival mucosa that could alter the colour of the area being shot and thus avoiding the need for post-processing the images to eliminate such areas of reflection.

According to an embodiment of the invention, the adapter/spacer can be removed and replaced with one having shape adapted to the individual patient, if the latter has a particularly different conformation of the periocular area than the norm, i.e. when the patient is very young and the periocular area is very small compared to the norm: in these cases, the shape and dimensions of the adapter can be further customised by specially designing and printing the adapter in a short time with low-cost technologies, such as 3D printing.

The device object of the present invention, thanks to the algorithms installed on, and implementable by, the computerised processing unit referred to in point a), is capable of estimating the haemoglobin concentration values in blood, by means of a calculation operation.

Said calculation operation, performed by the aforesaid algorithms starting from the selected portion of the image captured by means of the miniature video camera, is made accurate by means of a preliminary calibration of the same algorithms, which is performed during the designing step. According to a preferred aspect of the invention, this calibration consists in training a classifier, e.g. RUSBoost of a known type, used to determine whether the image of the conjunctiva belongs to a patient suffering from anaemia or not.

According to another aspect of the present invention, said software training can be performed not only upstream of the first use of the device but can also be repeated at subsequent times, in order to further increase the precision of the device dynamically during its use and the more frequently it is used.

Further object of the present invention is a method for the quick estimation of the haemoglobin concentration in a subject. Said method, which may be advantageously based on the use of said device, still according to the present invention, comprises the following steps:
i) displaying the palpebral conjunctiva and controlling the framing through at least one miniature video camera of the imaging unit of the device of the invention, by means of a display;
ii) capturing the image of the palpebral conjunctiva by means of photographic shooting of said miniature video camera;
iii) manually selecting or selecting in an assisted manner, on said display, the area of the photograph which shoots the palpebral conjunctiva;
iv) processing said photograph by the aforesaid algorithms, thereby obtaining a value that suggests any anaemic pathology in said subject.

Due to its simplicity, the use of the device and/or method according to the invention can also be performed by subjects without specific knowledge or training. In particular, in some cases it is the same subject who can employ the device for a self-measurement by following all the steps previously described.

According to an aspect of the present invention, the selection of the area of the photograph referred to in step iii) is initially made by the operator with less precise contours, by simply employing a finger or a suitable tool directly on the surface of the display and, subsequently, the software will proceed to delineate it more precisely thanks to the image analysis algorithms referred to in point a).

According to another aspect of the present invention, the selection of the area of the photograph referred to in step iii) is performed fully automatically by the device and the integrated software by identifying and selecting a conjunctiva image portion that the device deems sufficient to perform the calculations necessary to estimate the level of anaemia.

The operation of selecting the area occupied by the palpebral conjunctiva, on which the calculation of the values meanᵣ, mean_{g}, mean_{b}, mean_{L}*, meanₐ*, mean_{b}*, HHR, E, B, G₁, G₂, G₃, G₄, G₅ in the pixels will subsequently be performed, can be repeated, even several times, in case the operator does not consider it to have been performed correctly by the software.

According to an aspect of the present invention, step iv) provides for performing image analysis algorithms through extraction of information from said image and artificial intelligence algorithms for the estimation of the anaemia level in said subject to be evaluated.

In other words, the values meanᵣ, mean_{g}, mean_{b}, mean_{L}*, meanₐ*, mean_{b}*, HHR, E, B, G₁, G₂, G₃, G₄, G₅ in the pixels of the selected area are finally extracted and processed by the algorithms installed on, and implemented by, the computerised processing unit to obtain the estimation of the haemoglobin concentration and, consequently, the indication of a possible anaemic pathology.

The invention object of the present application allows to obtain numerous advantages compared to devices of the known art. In particular, the object of the invention is a stand-alone device which does not need to be coupled to other equipment in order to perform its function. In fact, the device according to the invention can be handled and correctly used even by non-healthcare personnel without special training. In some cases, it is the same subject who can employ the device for a self-measurement. Similarly, the method according to the present invention allows to evaluate the level of haemoglobin in the blood of a subject and thus to simultaneously evaluate a possible anaemic pathology by using the device of the invention, with the advantage of thus having available a fast, reliable, objective and reproducible method, which is not affected by subjective evaluations that could significantly influence the results and consequent evaluations.

The particular shape of the spacer/adapter mentioned in point e) allows the perfect adherence to the patient's periocular area and the total exclusion of ambient light inside the area that will be subjected to measurement, thus increasing the precision of the detection and its standardisation. These advantages are further guaranteed and implemented by the presence of the lighting unit that provides light of a constant and repeatable intensity and colour in the various measurements. The lighting unit of the present device further allows to minimise reflections due to moisture in the conjunctival mucosa, which appear in the image as white areas. The area shot by the imaging unit is consequently unaltered, thus eliminating the need for post-processing the images.

Moreover, the device of the invention does not need to have, within the palpebral conjunctiva image that is captured, the colour reference normally used in the technologies known so far. In fact, the particular shape of the spacer referred to in point e), allowing the perfect adherence to the periocular area of the patient, allows to exclude the influence of the ambient light inside the area that will be subjected to measurement and the inner illumination of the same spacer, which is obtained by means of simple LED bulbs mounted therein, allows to carry out both the training step and the actual measurement with the same type of illumination.

The device of the invention, in fact, is characterised by the absence of devices and/or systems and/or a colour table or a reference card to perform the comparison with standard colours during the capture of the image, in order to recalibrate the colour depending on the ambient light. This peculiarity allows the image to be used without changing the original colours, thus allowing a more reliable estimation.

Finally, the simple and compact structure of the device that is the object of the invention and its low cost make it easy to use and carry even in particular situations and/or remote locations where it would be difficult to perform laboratory analyses, such as those normally used for the determination of haemoglobinaemia in venous blood.

The device that is the object of the invention can be produced at very low costs and sold at much lower prices than, e.g., systems which provide for the use of smartphones, thus allowing for mass diffusion.

### Experimental Section

### Values Measured by the Software and Correlations

Assuming that the portion of the conjunctiva extracted for the analysis, ROI (Region Of Interest), is of N×M dimensions, then meanᵣ, mean_{g} and mean_{b} are respectively represented by the following equations [1-3]:

$${mean}_{r} = \frac{{\sum }_{i = 1}^{N} {\sum }_{j = 1}^{M} {Pixel}_{red} [ i ] [ j ]}{N * M} \qquad [ 1 ]$$

$${mean}_{g} = \frac{{\sum }_{i = 1}^{N} {\sum }_{j = 1}^{M} {Pixel}_{green} [ i ] [ j ]}{N * M} \qquad [ 2 ]$$

$${mean}_{b} = \frac{{\sum }_{i = 1}^{N} {\sum }_{j = 1}^{M} {Pixel}_{blue} [ i ] [ j ]}{N * M} \qquad [ 3 ]$$

wherein Pixel_{red}[i][j], Pixel_{green}[i][j] Pixel_{blue}[i][j] denote the red, green and blue components of Pixel[i] [j], respectively.

The erythema index (EI) is defined as:

$$E I = {mean}_{r} - {mean}_{g}$$

The values mean_{L}*, meanₐ* and mean_{b}* are extracted from the CIELAB colour space and represented by the following equations [4-6]:

$${mean}_{L}^{*} = \frac{{\sum }_{i = 1}^{N} {\sum }_{j = 1}^{M} {Pixel}_{L}^{*} [ i ] [ j ]}{N * M} \qquad [ 4 ]$$

$${mean}_{a}^{*} = \frac{{\sum }_{i = 1}^{N} {\sum }_{j = 1}^{M} {Pixel}_{a}^{*} [ i ] [ j ]}{N * M} \qquad [ 5 ]$$

$${mean}_{b}^{*} = \frac{{\sum }_{i = 1}^{N} {\sum }_{j = 1}^{M} {Pixel}_{b}^{*} [ i ] [ j ]}{N * M} \qquad [ 6 ]$$

wherein Pixel_{L}*[i][j], Pixelₐ*[i][j] Pixel_{b}*[i][j] denote the brightness (L*) and the chromatic coordinates (a* and b*) of Pixel[i][j], respectively.

The HHR (High Hue Ratio) value is expressed by the equation [7]:

$$H H R = \frac{{n}_{H}}{N} \qquad [ 7 ]$$

where n_{H} represents the number of high-hue pixels in an image that has a total number of pixels N. A high HHR value denotes a red area of greater amplitude. Consequently, the image that has a high HHR value identifies an image that does not show the presence of anaemia. The pixel threshold value for defining a pixel having high hue is set at 0.95.

The Entropy (E), calculated on the image converted to grayscale, is defined according to the equation [8]:

$$E = - {\sum }_{i} P ( {X}_{i} ) {log}_{2} P ( {X}_{i} ) \qquad [ 8 ]$$

where P(Xₜ) represents an estimated probability of occurrence of the i-th grey level Xₜ from the image histogram after the histogram equalisation. P(Xₜ) is a non-zero value. The B value is an index of brightness (the average grayscale of the image).

The values G₁, G₂, G₃, G₄, G₅ are useful for characterising the pixels in which blood vessels are present or not. First, the software calculates values based on the grey level, which depend on the differences between the grey level in the pixel considered and a statistical value representative of the surrounding area. In particular, considering the grayscale image of the segmented conjunctiva, it was observed that the blood vessels appear darker, have a lower grey level than the surrounding area. Therefore, a sliding window was identified on the grayscale image, with dimensions of 3×3 pixels and stop motion, to calculate said values for each pixel of the conjunctiva according to the following equations.

$${G}_{1} ( x , y ) = I ( x , y ) - {min}_{( s , t ) ∈ {S}_{x , y}^{3}} \{ I ( s , t ) \} \qquad [ 9 ]$$

The equation [9] represents the difference between the intensity level I(x,y) at the pixel with co-ordinates (x,y) and the minimum intensity recorded in the 3×3 window S³_{x,y} centred in the pixel with co-ordinates (x,y) and allows the blood vessel to be highlighted against the background.

$${G}_{2} ( x , y ) = {max}_{( s , t ) ∈ {S}_{x , y}^{3}} \{ I ( s , t ) \} - I ( x , y ) \qquad [ 10 ]$$

The equation [10] represents the difference between the maximum intensity within the 3×3 S³_{x,y} window considered and the intensity of the pixel with co-ordinates (x,y) to highlight the background against the blood vessel.

$${G}_{3} ( x , y ) = I ( x , y ) - {mean}_{( s , t ) ∈ {S}_{x , y}^{3}} \{ I ( s , t ) \} \qquad [ 11 ]$$

The equation [11] represents the difference between the intensity of the pixel with co-ordinates (x,y) and the average of the pixel intensities of the 3×3 S³_{x,y} window considered.

$${G}_{4} ( x , y ) = {std}_{( s , t ) ∈ {S}_{x , y}^{3}} \{ I ( s , t ) \} \qquad [ 12 ]$$

The equation [12] represents the standard deviation of the pixel intensities in the 3×3 window considered.

$${G}_{5} ( x , y ) = I ( x , y ) \qquad [ 13 ]$$

The equation [13] represents the intensity of the central pixel of the 3×3 window considered.

Once the five grayscale values have been calculated for each pixel, the software is able to apply the equation [14] as follows:

$${G}_{i} = {mean}_{i} g ( i ) \qquad [ 14 ]$$

$$where i = 1 , 2 , 3 , 4 , 5 .$$

### In Vivo Use of the Device According to the Invention

The device that is the object of the present invention has been shown to be capable of accurately estimating the presence of a possible anaemic pathology in a subject. Specifically, the software was trained, according to the RUSBoost procedure illustrated above, by using a set of images of the conjunctivas of 123 patients whose haemoglobin value had been concurrently measured in the laboratory by means of blood sampling according to normal, current laboratory protocols.

The threshold value established to define a patient in a “severe anaemia” condition corresponds to a haemoglobin (Hgb) value≤10.5 g/dL.

After the software training step of the device object of the present invention, measurements of the haemoglobin concentration were performed on subjects from whom a blood sample was taken to check the good quality of the measurement. The Pearson correlation index for the value of a* in the area of the palpebral conjunctiva with respect to the haemoglobin values measured by venous blood sampling was found to be 0.69, which corresponds to a strong correlation value, as stated in the literature. In order to provide an objective comparison, the same parameter was calculated by taking measurements on the same number of subjects and using the instrument described in IT102017000043624. In this case, a Pearson correlation index of 0.49 (moderate correlation) was derived for the value of a* in the area of the palpebral conjunctiva with respect to the haemoglobin values measured by means of venous blood sampling, which was significantly lower than that obtained thanks to the device of the invention.

1. A device for quickly and non-invasively estimating an anaemia condition in a subject to be evaluated, comprising a spacer configured, in use, to be contacted with a periocular area of said subject to be evaluated, said periocular area which comprises an eye of said subject; said spacer comprising a hollow body which comprises, in turn, a supporting surface configured, in use, to be contacted with said periocular area of said subject to be evaluated and a cavity extending longitudinally in said body starting from said supporting surface;
said device being characterised in that:
said supporting surface is configured to adapt to the shape of the frontal bone, zygomatic bone and maxillary bone of said subject to be evaluated;
and characterised in that it comprises:
an imaging unit combined with said cavity and comprising a camera configured to capture an image of said periocular area;
an optical unit combined with said imaging unit and comprising at least one lens;
a lighting unit arranged inside the spacer and combined with said cavity and configured to illuminate a space inside said cavity, preferably arranged in the proximity of the optical unit;
a display, preferably a LED display, for displaying said image;
a computerised processing unit operatively combined with said imaging unit and said lighting and display units, said computerised processing unit being configured to receive said image from said imaging unit and to process it, by implementing algorithms installed therein, in order to estimate the anaemia state of the subject to be evaluated and to send a result of the aforesaid processing to said display.

2. The device according to claim 1, wherein said body comprises a user surface opposite said supporting surface; said display being arranged at said user surface.

3. The device according to the preceding claim, wherein said cavity extends in said body from said supporting surface to said user surface; said display being arranged to close said cavity.

4. The device according to any one of the preceding claims, wherein the supporting surface is shaped or moulded to adapt and adhere to said periocular area of said subject to be evaluated.

5. Method for quickly and non-invasively estimating the anaemia state in a subject to be evaluated by using the device according to any one of the preceding claims, said method comprising the steps of:
i) displaying the palpebral conjunctiva and controlling the framing through at least one miniature video camera of the imaging unit of the device according to any one of the preceding claims, by means of a display;
ii) capturing an image of the palpebral conjunctiva by photographic shooting of said miniature video camera;
iii) selecting, on said display, an area of said image which shoots said area of the palpebral conjunctiva;
iv) processing said image by the aforesaid algorithms, thus obtaining an estimated value or estimation of the level of anaemia in said subject to be evaluated.

6. Method according to the preceding claim, wherein said subject to be evaluated employs the device for a self-measurement.

7. Method according to claim 5 or 6, wherein step iii) provides for manually selecting on said display said area of said image which shoots said area of the palpebral conjunctiva.

8. Method according to any one of claims 5 to 7, wherein step iv) provides for performing image analysis algorithms for the extraction of information from said image and artificial intelligence algorithms for the estimation of the anaemia level in said subject to be evaluated.
